(19) United States
(12) Patent Application Publication
(10) Pub. No.: US 2024/0264083 A1
(43) Pub. Date: Aug. 8, 2024

(54) TECHNIQUE FOR QUANTITATIVE DETECTION OF BETA-GALACTOSIDASE (BETA-GAL) IN SEAWATER BASED ON SURFACE-ENHANCED RAMAN SPECTROSCOPY (SERS)

(71) Applicant: SHANGHAI OCEAN UNIVERSITY, Shanghai (CN)

(72) Inventors: Jiasong FANG, Shanghai (CN); Yong WU, Shanghai (CN); Hongge ZHANG, Shanghai (CN); Tianqiang BAO, Shanghai (CN); Junwei CAO, Shanghai (CN)

(21) Appl. No.: 18/041,023

(22) PCT Filed: Oct. 25, 2022
(86) PCT No.: PCT/CN2022/127315

§ 371 Date: Feb. 8, 2023

(30) Foreign Application Priority Data
Dec. 22, 2021 (CN) 202111583690.4

(51) Int. Cl. G01N 21/65; G01N 33/18

(52) U.S. Cl. CPC G01N 21/658; G01N 33/18; G01N 2333/938

(57) ABSTRACT

The present disclosure discloses a technique for quantitative detection of β-galactosidase (β-GAL) based on surface-enhanced Raman spectroscopy (SERS), including the following steps: a. taking 180 μL of each of a 5-bromo-4-chloro-3-indolyl β-D-galactoside (BCIG) solution, a β-GAL solution, and dimethylsulfoxide (DMSO); b. preparing a plurality of β-GAL samples with different activities in advance; c. adding an equal volume of a colloidal gold nanoparticle dropwise to each of the plurality of standard solutions with different activities, and conducting SERS; d. mixing a seawater sample to be tested with BCIG, incubating a resulting mixture to allow a reaction, adding DMSO and a colloidal gold nanoparticle, and directly detecting SERS signals of a product and the DMSO; and e. comparing the SERS signals obtained in step d with the standard curve to obtain an activity of the seawater sample to be tested.

### CROSS REFERENCE TO RELATED APPLICATION

The present application claims priority to Chinese Patent CN202111583690.4 filed to the China National Intellectual Property Administration (CNIPA) on Dec. 22, 2021 and entitled “A Technique FOR QUANTITATIVE DETECTION OF β-GALACTOSIDASE (β-GAL) IN SEAWATER BASED ON SURFACE-ENHANCED RAMAN SPECTROSCOPY (SERS)”, which is incorporated herein by reference in its entirety.

### TECHNICAL FIELD

The present disclosure belongs to the technical field of Raman spectroscopy detection, and specifically relates to a technique for quantitative detection of β-galactosidase (β-GAL) in seawater based on surface-enhanced Raman spectroscopy (SERS).

### BACKGROUND

Oceans cover 71% of the Earth's surface area, and marine ecosystems are the largest ecosystems on the Earth. In a marine environment, dissolved organic matter (DOM) produced by primary productivity and DOM of other sources mostly exist in polymeric form, which cannot be directly utilized by microorganisms, and needs to be first decomposed by extracellular enzymes into low-molecular weight monomers or polymers with a molecular weight of lower than 700 Daltons (Da) and then absorbed by microorganisms. Marine microorganisms are major participants in the above process, and marine microorganism-mediated enzymatic hydrolysis of organic matter is key to limiting the mutual transformation of DOM and particulate organic matter (POM). Monomeric compounds released during hydrolysis can also be utilized by microorganisms to maintain metabolism. Thus, microorganisms not only serve as decomposers and producers, but also beneficiaries. The activity of a microbial extracellular enzyme is a core driver of the whole process, and plays a decisive role in cycling rate and spatial distribution of marine carbon. The study of marine extracellular enzymes is of great significance for the comprehensive understanding of material cycle, energy cycle, and microbial life activities of marine ecosystems and the evaluation of water quality and ecological efficiency.

β-GAL (scientific name: β-D-galactosidase, β-GAL) is a hydrolase capable of specifically hydrolyzing β-1,4-glycosidic bonds, and hydrolyzing lactose into glucose and galactose. β-GAL is widely present in marine waters. Marine plants, bacteria, fungi, and the like can produce β-GAL and release β-GAL into the surrounding water. β-GAL can decompose polysaccharides in seawater into monosaccharides or small-molecule polysaccharides of lower than 700 Da to maintain the metabolism of these organisms. The metabolism and death of these organisms can provide a large amount of organic carbon (OC) for the surrounding water. Therefore, the activity of β-GAL not only reflects the population structure of marine microorganisms and the distribution of marine organic nutrients, but also has great significance for revealing the mechanism of marine microorganisms in driving marine carbon cycle. Common methods for detecting the activity of β-GAL in seawater include the fluorescent simulated substrate method, spectrophotometry methods, and the like, which involve complicated sample pre-treatment and time-consuming detection. However, the activity of an extracellular enzyme is very easily affected by various factors including temperature, pressure, pH, and oxygen content, and thus a method for rapidly and efficiently detecting the activity of an extracellular enzyme is urgently needed.

Laser Raman spectroscopy (LRS) is an inelastic scattering phenomenon caused by energy exchange between a laser photon and a molecule of a substance when light irradiates a surface of the substance. LRS can reflect the internal energy level structure of the molecule of the substance and provide the molecular vibration information. SERS makes use of the optical enhancement effect of a metal nanoparticle such as gold and silver to enhance a Raman spectrum signal of a target molecule adsorbed on the particle, thereby achieving the rapid detection of a low-concentration substance. In recent years, due to advantages such as high efficiency, simple sample pre-treatment, non-damage, and non-contact, SERS has been widely used in food safety, biological detection, and other fields.

### SUMMARY

To overcome the above problems, the present disclosure provides a technique for quantitative detection of β-GAL in seawater based on SERS.

The present disclosure provides a technique for constructing a quantification model for quantitative detection of β-GAL in seawater, including the following steps:
(1) preparing a plurality of β-GAL samples with different activities in advance, mixing each of the β-GAL samples with a 5-bromo-4-chloro-3-indolyl β-D-galactoside (BCIG) solution, incubating resulting mixtures for a specified period of time, and then adding DMSO to obtain a plurality of standard solutions with different β-GAL activities; and
(2) adding an equal volume of a colloidal gold nanoparticle dropwise to each of the plurality of standard solutions with different β-GAL activities, conducting SERS, and plotting a standard curve according to a relationship between a relative intensity of an SERS signal of each of the plurality of standard solutions with different β-GAL activities against an SERS signal of DMSO and a logarithm value of an activity of the standard solution, where the standard curve is the quantification model.

The present disclosure provides a technique for quantitative detection of β-GAL in seawater based on SERS, including the following steps:
S1. mixing a seawater sample to be tested with BCIG, incubating a resulting mixture to allow a reaction, and adding DMSO and a colloidal gold nanoparticle; and directly detecting SERS signals of a product and the DMSO, and calculating an SERS signal ratio of the product to the DMSO, where the product is 5-bromo-4-chloro-3-indole (BCI) oxidized dimer; and
S2. substituting the SERS signal ratio of the product to the DMSO obtained in S1 into a quantification model obtained by the construction technique described in the above technical solution to obtain a β-GAL activity of the seawater sample to be tested.

The present disclosure also provides a technique for quantitative detection of β-GAL in seawater based on SERS, including the following steps:
a. taking 180 μL of each of a BCIG solution, a β-GAL solution, DMSO, and a solution obtained after a reaction of BCIG and β-GAL, adding 180 μL of DMSO, adding 200 μL of a colloidal gold nanoparticle (70 nm), and subjecting each of resulting mixtures to SERS analysis on a machine (SERS spectra of the solvent and substrate used in the experiment serve as blank groups, and experimental results show that a Raman spectral peak at 600 cm⁻¹ is a characteristic peak of the product (BCI oxidized dimer.) and a Raman spectral peak at 677 cm⁻¹ is a characteristic peak of DMSO and can serve as an internal standard peak of this experiment);
b. preparing a plurality of β-GAL samples with different activities in advance, mixing each of the β-GAL samples with a BCIG solution, incubating resulting mixtures for a specified period of time, and then adding DMSO to obtain a plurality of standard solutions with different β-GAL activities (The β-GAL solutions with different activities are prepared to establish a quantification standard curve. On the one hand, DMSO can dissolve the product to make the solution homogeneous, thereby improving the stability and reliability of detection; and on the other hand, DMSO can serve as an internal standard to make the quantification model stable);
c. adding an equal volume of a colloidal gold nanoparticle dropwise to each of the plurality of standard solutions with different β-GAL activities, conducting SERS, and plotting a standard curve according to a relationship between a relative intensity of an SERS signal of each of the plurality of standard solutions with different β-GAL activities against an SERS signal of DMSO and a logarithm value of an activity of the standard solution (The intensities of a product target peak and a DMSO internal standard peak are enhanced, and a quantification standard curve is established according to a relationship between the two);
d. mixing a seawater sample to be tested (seawater usually includes β-GAL) with BCIG, incubating a resulting mixture to allow a reaction, and adding DMSO and a colloidal gold nanoparticle; and directly detecting SERS signals of a product and the DMSO, and calculating an SERS signal ratio of the product to the DMSO, where the product is BCI oxidized dimer (the sensitivity of the technique is required to meet the requirements for detection of an activity of β-GAL in seawater); and
e. comparing the SERS signal ratio of the product to the DMSO obtained in step d with the standard curve to obtain an activity of the seawater sample to be tested (The above standard curve is used to quantitatively detect an activity of β-GAL in the seawater sample).

Further, the colloidal gold nanoparticle has a particle size of 70 nm.

Further, the SERS signal of the DMSO refers to a peak intensity of the DMSO at a Raman shift of 677 cm⁻¹.

Further, the SERS signal of the β-GAL refers to a peak intensity of the β-GAL at a Raman shift of 600 cm⁻¹. Steps d and e are conducted to detect an activity of β-GAL in seawater, where on the one hand, it is necessary to determine whether there is β-GAL in the seawater sample, and on the other hand, the obtained standard curve is used to conduct quantitative analysis of the activity of β-GAL in the seawater sample.

Further, β-GAL activity data of the standard solutions with different β-GAL activities have an average relative standard deviation (RSD) of less than 15%, indicating that the technique has prominent stability.

Further, an average value of β-GAL activity data of the standard solutions with different β-GAL activities refers to a peak intensity at a Raman shift of 600 cm⁻¹/a peak intensity at a Raman shift of 677 cm⁻¹ (abbreviated as 600 cm⁻¹/677 cm⁻¹).

Further, an ordinary least squares (OLS) method is used to linearly fit a β-GAL concentration and an SERS intensity ratio in the standard curve to obtain a standard equation; and
the SERS intensity ratio refers to a ratio of the SERS signal to the SERS signal of the DMSO.

Further, the fitted standard equation of the standard curve in step c is y=0.784*x+0.004, with a correlation coefficient R²=0.936, where x represents a logarithm value of an activity of a β-GAL-active standard solution and y represents a ratio of an SERS signal of the β-GAL-active standard solution to the SERS signal of the DMSO.

### Beneficial Effects

The present disclosure uses LRS for the first time to detect an activity of β-GAL in seawater, which has sensitivity meeting requirements for detection of β-GAL in seawater.

The technique of the present disclosure has an average RSD of less than 15%, indicating excellent reliability; and the introduction of DMSO as an internal standard improves the stability of detection.

The technique has advantages such as no sample pre-treatment (a substrate is added to allow a reaction for a specified period of time, then an internal standard and a surface-reinforced gold nanoparticle are added, and then detection is directly conducted), non-damage (the excitation light is visible light and causes no damage to a sample), and rapid detection (compared with the fluorescence method that takes more than ten hours or even tens of hours for detection, the technique of the present disclosure only takes a few hours and thus is suitable for in situ detection).

The technique of the present disclosure provides a strong scientific theoretical support for the in situ quantitative detection of an activity of an extracellular enzyme in seawater.

### BRIEF DESCRIPTION OF THE DRAWINGS

FIG. 1 shows a reaction formula of hydrolysis of BCIG by β-GAL;

FIG. 2 shows SERS spectra of the experimental solvent and substrate;

FIG. 3 shows SERS spectra corresponding to different β-GAL activities;

FIG. 4 shows the linear equation fitting of a standard curve; and

FIG. 5 shows an SERS spectrum of a seawater sample after reacting with BCIG.

### DETAILED DESCRIPTION OF THE EMBODIMENTS

The embodiments of the present disclosure will be described in detail below in conjunction with accompanying drawings. The embodiments are implemented on the premise of the technical solutions of the present disclosure, and detailed implementations and specific operation processes are provided, but the protection scope of the present disclosure is not limited to the following embodiments.

Detection principle of the present disclosure:

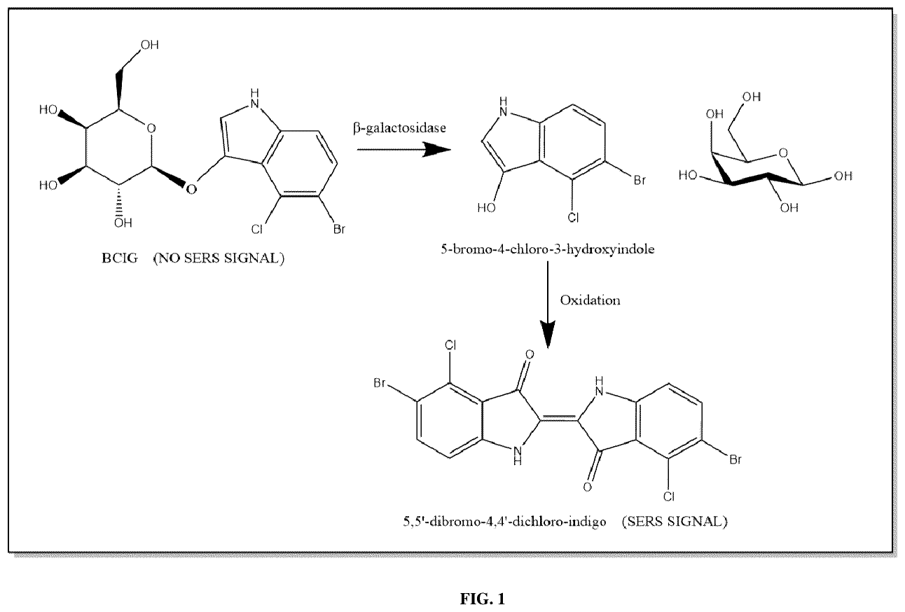

β-GAL can specifically catalyze the hydrolysis of β-1,4-glycosidic bonds, and a principle of the hydrolysis is shown in FIG. 1. β-GAL hydrolyzes BCIG without SERS characteristics to obtain BCI, and BCI is rapidly oxidized into a water-insoluble BCI oxidized dimer with strong SERS characteristics; and a β-GAL activity is determined by establishing a relationship between different β-GAL activities and intensities of SERS characteristic peaks of products.

Theoretically, a Raman spectral characteristic peak of the product BCI oxidized dimer appears at around 600 cm⁻¹, which is verified by an experiment below.

200 μL of a BCIG solution with a concentration of 2 mg/mL, 200 μL of a β-GAL solution with a concentration of 5 U/L, 200 μL of DMSO, and a solution obtained after a reaction of a BCIG solution (20 μL, 2 mg/mL) and a β-GAL solution (180 μL, 5 U/L) for 4 h each are taken and added to 4 sample bottles, respectively, 200 μL of a colloidal gold nanoparticle is added to each sample bottle, and resulting mixtures each are thoroughly mixed and subjected to SERS (excitation wavelength: 785 nm, excitation time: 10 s, and excitation frequency: 5 mW, where 5 Raman spectra are collected for each sample).

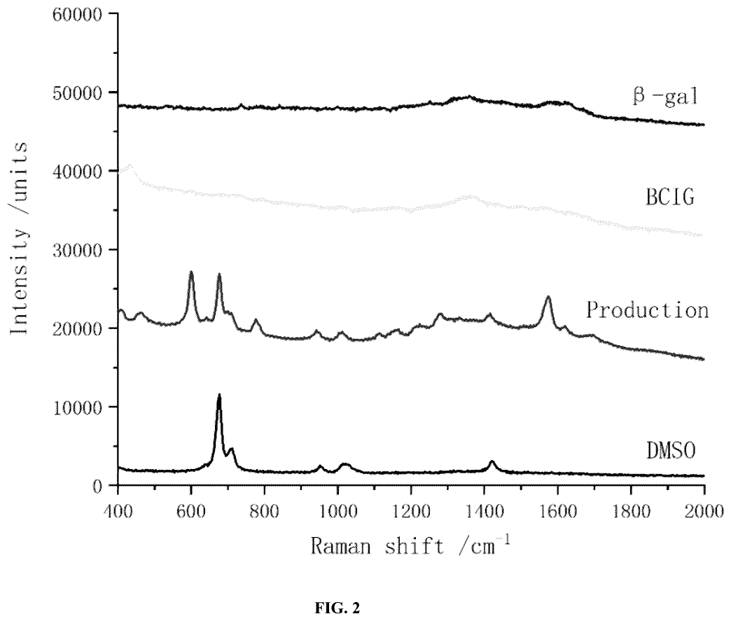

As shown in FIG. 2, the BCIG solution and the β-GAL solution do not have SERS characteristics, and DMSO does not have a Raman spectral peak at around 600 cm⁻¹ and has two obvious SERS characteristic peaks at around 677 cm⁻¹ and 700 cm⁻¹, which represent a C—S—C symmetrical stretching vibration peak and a C—S stretching vibration peak, respectively; and the Raman spectral peak at 677 cm⁻¹ is selected as an internal standard peak for quantitative analysis of the activity of the extracellular enzyme (β-GAL). A BCI oxidized dimer obtained after a reaction of BCIG and β-GAL for 4 h has a strong SERS peak at 600 cm⁻¹, and the SERS peak is a Raman peak caused by plane vibration of C═C—CO—C in a chemical structure of the product, which is a characteristic peak of the product.

### Example 1 Quantification Model

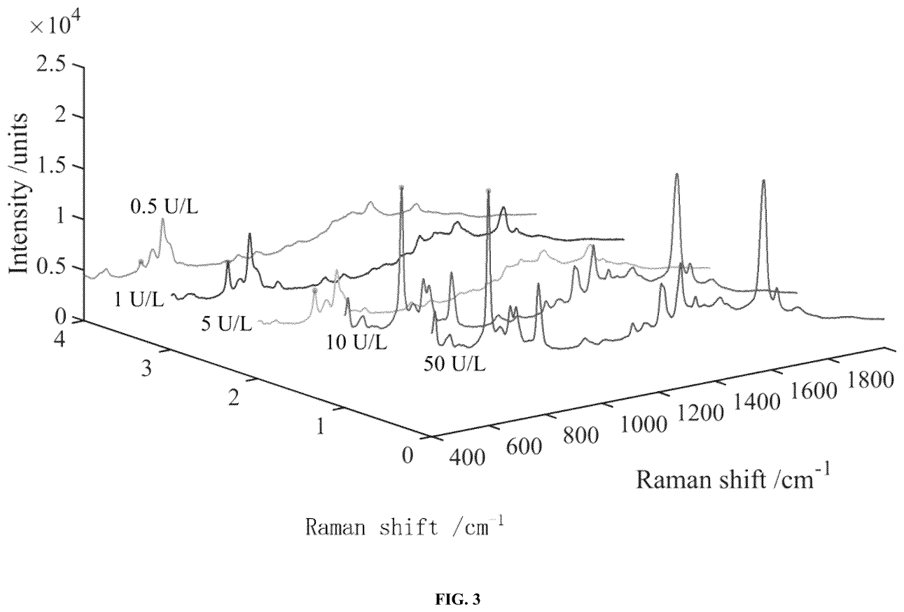

5 β-GAL solutions with different activities (50 U/L, 10 U/L, 5 U/L, 1 U/L, and 0.5 U/L) of 900 μL each were prepared, and 5 parallel samples were set for each activity; each of the samples was mixed with 100 μL of a BCIG solution with a concentration of 2 mg/mL, and a resulting mixture was incubated for 4 h; and then 180 μL of a resulting reaction solution was taken, 20 μL of DMSO was added, and an SERS signal was tested. Resulting spectra were shown in FIG. 3.

It can be seen from FIG. 3 that there is no direct linear relationship between an intensity of an SERS characteristic peak of the BCI oxidized dimer and the concentration of β-GAL. Because an intensity of a Raman spectrum is disturbed by factors such as laser power stability, enhanced reagent homogeneity, and solvent background noise, it is difficult to achieve quantitative analysis directly with an intensity of a characteristic peak of a Raman spectrum. Therefore, a Raman spectral characteristic peak of the added DMSO at around 677 cm⁻¹ was used as an internal standard peak, and a quantitative detection model was established by the internal standard method to achieve the quantitative detection of β-GAL. Table 1 shows the SERS characteristic peak intensity information for substrates and internal standards.

TABLE 1
Characteristic peak intensities of substrates and internal standards

| β-GAL | Average | Average | Average intensity | |
|---|---|---|---|---|
| activity/ | intensity/ | intensity/ | ratio/ | RSD/ |
| (U/L) | (600 cm⁻¹) | (677 cm⁻¹) | (600/677) | (600/677) |
| 50 | 143171 | 14193 | 2.017 | 3.59% |
| 10 | 123521 | 13374.2 | 1.847 | 10.3% |
| 5 | 12956.6 | 10850.6 | 1.194 | 0.80% |
| 1 | 9427 | 12436.6 | 0.758 | 1.30% |
| 0.5 | 5923.8 | 10713.4 | 0.553 | 0.92% |

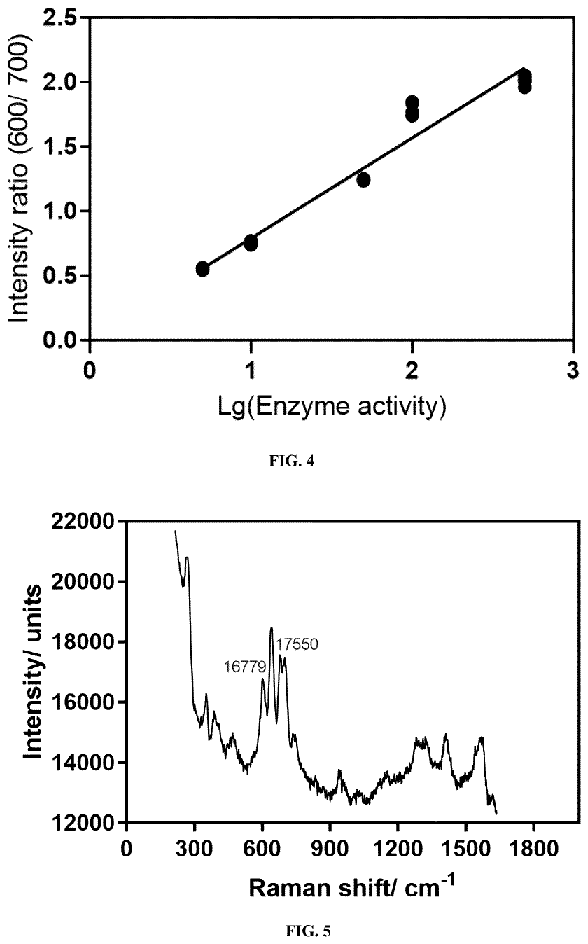

It could be seen from Table 1 that, overall, an intensity of the product characteristic peak (600 cm⁻¹) and an intensity of the internal standard peak (677 cm⁻¹) gradually decreased with the decrease of β-GAL activity, but there was no prominent functional relationship between the intensity and the activity. An RSD of intensity ratios corresponding to each enzyme activity is less than 10%, indicating that the SERS data have high reliability. An OLS method was used to linearly fit a β-GAL concentration and an SERS intensity ratio (600 cm⁻¹/677 cm⁻¹), as shown in FIG. 4.

DMSO was introduced as an internal standard, and a characteristic peak of DMSO at 677 cm⁻¹ was used as an internal standard peak. With a logarithm of each of 5 β-GAL activities (50 U/L, 10 U/L, 5 U/L, 1 U/L, and 0.5 U/L) as an abscissa and a ratio of a product characteristic peak (600 cm⁻¹) to the internal standard peak (677 cm⁻¹) as an ordinate, the following standard equation was fitted: y=0.784*x+0.004, with a correlation coefficient R²=0.936, and it could be known that there was a strong linear relationship between the β-GAL activity and the Raman spectral characteristic peak intensity ratio (600 cm⁻¹/677 cm⁻¹). The model had the ability to quantitatively detect a β-GAL activity.

### Example 2 Seawater Verification Test

Fresh seawater samples were collected from the East China Sea (30° 39′48″N, 122° 29′48″E) in December 2020. The samples were surface seawater from the ocean and were directly collected by a fishing vessel. 900 μL of a fresh seawater sample was mixed with 100 μL of a BCIP solution with a concentration of 2 mg/mL, and a resulting mixture was incubated for 4 h; and then 180 μL of a resulting reaction solution was taken, 20 μL of DMSO was added, and an SERS signal was tested. Resulting spectra were shown in FIG. 5.

As shown in FIG. 5, a significant Raman spectral peak (peak intensity: 16,779) appeared at 600 cm⁻¹, indicating that the presence of β-GAL in seawater was successfully determined by this technique; the Raman spectral intensity at 677 cm⁻¹ reached 17,550, which was a Raman spectral peak caused by symmetrical stretching vibration of C—S—C in DMSO; and a ratio of the two peaks was 0.956, and the ratio value was substituted into the above model to achieve quantitative detection of the β-GAL activity of the seawater sample, where the determined β-GAL activity value of the seawater sample was equivalent to the commercial β-GAL activity of 0.824 U/L.

With BCIG as a substrate and DMSO as an internal standard, a technique for quantitatively detecting a β-GAL activity in seawater based on SERS was proposed. The results showed that there was a prominent linear relationship between the β-GAL activity and the intensity ratio of the characteristic peak to the internal standard peak (600 cm⁻¹/677 cm⁻¹), and a correlation coefficient was 0.936. With the model, the β-GAL activity in the seawater sample was successfully quantitatively detected, and the rapid detection of β-GAL activity in seawater was realized. In addition, the technique can also be used in the detection of activities of extracellular enzymes of other microorganisms in seawater, which lays a solid scientific foundation for the in situ detection of activities of microbial extracellular enzymes in seawater.

The above description of embodiments is merely provided to help illustrate the technique of the present disclosure and a core idea thereof. It should be noted that several improvements and modifications may be made by persons of ordinary skill in the art without departing from the principle of the present disclosure, and these improvements and modifications should also fall within the protection scope of the present disclosure. Various modifications to these embodiments are apparent to those of professional skill in the art, and the general principles defined herein may be implemented in other embodiments without departing from the spirit or scope of the present disclosure. Thus, the present disclosure is not limited to the embodiments shown herein, but falls within the widest scope consistent with the principles and novel features disclosed herein.

What is claimed is:

1. A technique for constructing a quantification model for quantitative detection of β-galactosidase (β-GAL) in seawater, comprising the following steps:
(1) preparing a plurality of β-GAL samples with different activities in advance, mixing each of the β-GAL samples with a 5-bromo-4-chloro-3-indolyl β-D-galactoside (BCIG) solution, incubating resulting mixtures for a specified period of time, and then adding dimethylsulfoxide (DMSO) to obtain a plurality of standard solutions with different β-GAL activities; and
(2) adding an equal volume of a colloidal gold nanoparticle dropwise to each of the plurality of standard solutions with different β-GAL activities, conducting surface-enhanced Raman spectroscopy (SERS), and plotting a standard curve according to a relationship between a relative intensity of an SERS signal of each of the plurality of standard solutions with different β-GAL activities against an SERS signal of DMSO and a logarithm value of an activity of the standard solution, wherein the standard curve is the quantification model.

2. A technique for quantitative detection of β-GAL in seawater based on SERS, comprising the following steps:
S1. mixing a seawater sample to be tested with BCIG, incubating a resulting mixture to allow a reaction, and adding DMSO and a colloidal gold nanoparticle; and directly detecting SERS signals of a product and the DMSO, and calculating an SERS signal ratio of the product to the DMSO; and
S2. substituting the SERS signal ratio of the product to the DMSO obtained in S1 into a quantification model obtained by the technique according to claim 1 to obtain a β-GAL activity of the seawater sample to be tested.

3. A technique for quantitative detection of β-GAL in seawater based on SERS, comprising the following steps:
a. taking 180 μL of each of a BCIG solution, a β-GAL solution, DMSO, and a solution obtained after a reaction of BCIG and β-GAL, adding 180 μL of DMSO, adding 200 μL of a colloidal gold nanoparticle, and subjecting each of resulting mixtures to SERS analysis on a machine;
b. preparing a plurality of β-GAL samples with different activities in advance, mixing each of the β-GAL samples with a BCIG solution, incubating resulting mixtures for a specified period of time, and then adding DMSO to obtain a plurality of standard solutions with different β-GAL activities;
c. adding an equal volume of a colloidal gold nanoparticle dropwise to each of the plurality of standard solutions with different β-GAL activities, conducting SERS, and plotting a standard curve according to a relationship between a relative intensity of an SERS signal of each of the plurality of standard solutions with different β-GAL activities against an SERS signal of DMSO and a logarithm value of an activity of the standard solution;
d. mixing a seawater sample to be tested with BCIG, incubating a resulting mixture to allow a reaction, and adding DMSO and a colloidal gold nanoparticle; and directly detecting SERS signals of a product and the DMSO, and calculating an SERS signal ratio of the product to the DMSO; and
e. comparing the SERS signal ratio of the product to the DMSO obtained in step d with the standard curve to obtain a β-GAL activity of the seawater sample to be tested.

4. The technique according to claim 1, wherein the colloidal gold nanoparticle has a particle size of 70 nm.

5. The technique according to claim 1, wherein the SERS signal of the DMSO refers to a peak intensity of the DMSO at a Raman shift of 677 cm⁻¹.

6. The technique according to claim 1, wherein the SERS signal of the β-GAL refers to a peak intensity of the β-GAL at a Raman shift of 600 cm⁻¹.

7. The technique according to claim 1, wherein β-GAL activity data of the standard solutions with different β-GAL activities have an average relative standard deviation (RSD) of less than 15%.

8. The technique according to claim 1, wherein an average value of β-GAL activity data of the standard solutions with different β-GAL activities refers to a peak intensity at a Raman shift of 600 cm⁻¹/a peak intensity at a Raman shift of 677 cm⁻¹.

9. The technique according to claim 1, wherein an ordinary least squares (OLS) method is used to linearly fit a β-GAL concentration and an SERS intensity ratio in the standard curve to obtain a standard equation; and
the SERS intensity ratio refers to a ratio of the SERS signal to the SERS signal of the DMSO.

10. The technique according to claim 1, wherein the fitted standard equation of the standard curve is y=0.784*x+0.004, with a correlation coefficient R²=0.936, wherein x represents a logarithm value of an activity of a β-GAL-active standard solution and y represents a ratio of an SERS signal of the β-GAL-active standard solution to the SERS signal of the DMSO.

11. The technique according to claim 2, wherein the colloidal gold nanoparticle has a particle size of 70 nm.

12. The technique according to claim 3, wherein the colloidal gold nanoparticle has a particle size of 70 nm.

13. The technique according to claim 2, wherein the SERS signal of the DMSO refers to a peak intensity of the DMSO at a Raman shift of 677 cm⁻¹.

14. The technique according to claim 3, wherein the SERS signal of the DMSO refers to a peak intensity of the DMSO at a Raman shift of 677 cm⁻¹.

15. The technique according to claim 2, wherein the SERS signal of the β-GAL refers to a peak intensity of the β-GAL at a Raman shift of 600 cm⁻¹.

16. The technique according to claim 3, wherein the SERS signal of the β-GAL refers to a peak intensity of the β-GAL at a Raman shift of 600 cm⁻¹.

17. The technique according to claim 3, wherein β-GAL activity data of the standard solutions with different β-GAL activities have an average relative standard deviation (RSD) of less than 15%.

18. The technique according to claim 3, wherein an average value of β-GAL activity data of the standard solutions with different β-GAL activities refers to a peak intensity at a Raman shift of 600 cm⁻¹/a peak intensity at a Raman shift of 677 cm⁻¹.

19. The technique according to claim 3, wherein an ordinary least squares (OLS) method is used to linearly fit a β-GAL concentration and an SERS intensity ratio in the standard curve to obtain a standard equation; and
the SERS intensity ratio refers to a ratio of the SERS signal to the SERS signal of the DMSO.

20. The technique according to claim 3, wherein the fitted standard equation of the standard curve is y=0.784*x+0.004, with a correlation coefficient R²=0.936, wherein x represents a logarithm value of an activity of a β-GAL-active standard solution and y represents a ratio of an SERS signal of the β-GAL-active standard solution to the SERS signal of the DMSO.
